(19) United States
(12) Patent Application Publication
(10) Pub. No.: US 2008/0033572 A1
(43) Pub. Date: Feb. 7, 2008

(54) Bone graft composites and methods of treating bone defects

(75) Inventors: Paul D'Antonio, Morristown, NJ (US); Joshua Simon, Rockaway, NJ (US); Benjamin T. Cripps, Teaneck, NJ (US)

Correspondence Address: HARNESS, DICKEY & PIERCE, P.L.C., P.O. BOX 828, BLOOMFIELD HILLS, MI, 48303, US

(73) Assignee: EBI L.P., Parsippany, NJ (US)

(21) Appl. No.: 11/498,341

(22) Filed: Aug. 3, 2006

(51) Int. Cl. A61F 2/28

(52) U.S. Cl. 623/23.51; 623/23.63; 606/77

(57) ABSTRACT

Bone graft compositions comprising demineralized bone matrix, calcium phosphate, collagen and bioinductive cellular solution. Methods to repair and heal defective and missing bone using the bone graft compositions are also described.

### BACKGROUND

The present invention generally relates to compositions and methods of making bone composites to treat (osseous) bone defects.

Bone graft materials should be designed with the physiological processes innately involved in bone formation and remodeling in mind. Osteogenesis and repairing of bone defects is a complex biological process requiring the concerted actions of bone forming cells such as osteoblasts and bone resorptive cells such as osteoclasts. In the case of a fracture or bone disease or defect, proper bone healing and subsequent bone remodeling is highly dependent on maintaining compatibility between osteoconductive materials that form the framework of the bone replacement and the osteoinductive materials which initiate replacement of the bone replacement with natural bone.

Current bone graft materials include autografts (bone material obtained from the patient), allografts (the use of cadaver bone and bone material), xenografts (bone materials from animals), and a variety of artificial or synthetic bone substitute materials. Bone grafting techniques employing allograft or autograft materials possess intrinsic high biocompatibility, however the harvest of autogenous bone results in high patient morbidity and presents an increased risk of infection. Costs associated with autograft bone replacements often make this technique prohibitive, however, when compared to the use of synthetic premanufactured bone grafts.

Synthetic graft materials are also used in repair of bone defects. However, such materials may present issues of biocompatibility and efficacy. In particular, the defect site containing a synthetic bone replacement must be nourished through direct blood supply and transfusion of body fluids. The distribution of the sizes of open connected pores within the bone graft is an important variable among others in the success of tissue repair and bone in-growth, as such physical characteristics influence the cellular processes involved in healthy bone healing. Optimal pore size distribution within the bone graft enables and enhances bone remodeling and associated sequelae, including cell seeding, vascular in-growth, and bone resorption and replacement of the artificial bone graft material with natural bone.

The synthetic bone graft material should, in addition to supporting the chemotaxis of osteogenic cells from neighboring bone tissue, provide and support a resident population of progenitor stem cells that can differentiate into osteocytes. The bone graft material should provide osteogenic stimuli to the resident osteocytes to express factors that will enhance the deposition and remodeling of new bone.

It is desirable to have a new biocompatible synthetic bone graft material that allows the in-growth of bone cells that promote osteogenesis. The ideal bone graft material will also have a resident population of bone progenitor cells which can be isolated and incorporated into the bone graft intraoperatively, thus allowing the embedded stem cells to differentiate into bone forming cells to enhance the rate of bone replacement and bone remodeling.

### SUMMARY

The present teaching provides bone graft matrix compositions and methods of using bone graft matrix compositions to treat osseous voids and defects. In various embodiments, the compositions comprise:
demineralized bone matrix (DBM);
calcium phosphate;
collagen; and
bioinductive cellular solution.
   In various embodiments, the compositions optionally comprise biologically active factors to improve the osteoinductive potential of the bone graft matrix. Biologically active factors include growth factors, structural proteins including fibronectins and laminins, cytokines, antibiotics, chemotherapeutics and serum proteins.

In various embodiments, methods are provided to produce continuous non-moldable, non-flowable bone graft scaffolds for the replacement of diseased or otherwise defective bone comprising expandable matrices formed preoperatively or intraoperatively and implanted during surgery. In various embodiments, methods for producing bone graft composite materials are described comprising progenitor cells after adding blood, bone marrow, adipose tissue, liposuction aspirate, and combinations thereof. In various embodiments, methods are described for producing bone graft materials having the consistency of putty so as to be moldable, for example, when inserted into a bone void or incorporated into an orthopedic device.

### DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Introduction” and “Summary”) and sub-headings (such as “Bone Graft Applications”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof.

The citation of any references herein or during prosecution of this application herein does not constitute an admission that those references are prior art or have any relevance to the patentability of the invention disclosed herein. Any discussion of the content of references is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features. Specific examples are provided for illustrative purposes of how to make, use and practice the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

As used herein, the term “about,” when applied to the value for a parameter of a composition or method of this invention, indicates that the calculation or the measurement of the value allows some slight imprecision without having a substantial effect on the chemical or physical attributes of the composition or method.

As referred to herein, the terms “a” and “an” mean at least one.

### Bone Graft Compositions

In various embodiments, the present invention provides compositions comprising:
demineralized bone matrix (DBM);
calcium phosphate;
collagen; and
bioinductive cellular solution.
   The compositions and methods are provided for the treatment of bone defects in human or other animal subjects. Specific compounds and compositions to be used in the invention must, accordingly, be biomedically acceptable. As used herein, such a “biomedically acceptable” component is one that is suitable for use with humans and/or animals without undue adverse side effects (such as toxicity, irritation, and allergic response) commensurate with a reasonable benefit/risk ratio.

In various embodiments, the bone graft composition is formulated using calcium phosphate ceramic, demineralized bone matrix and collagen, provided in powdered form which is then hydrated into an expandable matrix or putty by hydrating the powdered bone graft composition with one or more bioinductive cellular solutions comprising one or more of blood, bone marrow aspirate, or adipose tissue liposuction aspirate.

### Calcium Phosphate Ceramics

The compositions of the present invention comprise a calcium phosphate ceramic. In certain embodiments of the present teachings, calcium phosphate ceramics are chemically compatible to that of the mineral component of bone tissues. Examples of such calcium phosphate ceramics include calcium phosphate compounds and salts, and combinations thereof, including:
tricalcium phosphate Ca₃(PO₄)₂ (TCP), including alpha-TCP, beta-TCP, and biphasic calcium phosphate containing alpha- and beta-TCP;
amorphous calcium phosphate (ACP);
monocalcium phosphate Ca(H₂PO₄)₂ (MCP) and monocalcium phosphate monohydrate Ca(H₂PO₄)₂.H₂O (MCPM);

dicalcium phosphate CaHPO₄ (DCP) and dicalcium phosphate dihydrate CaBPO₄.2H₂O (DCPD);
tetracalcium phosphate Ca₄(PO₄)₂O (TTCP);
octacalcium phosphate Ca₈(PO₄)₄(HPO₄)₂.5H₂O (OCP);
calcium hydroxyapatite Ca₁₀(PO₄)₆(OH)₂ (CHA);
calcium oxyapatite Ca₁₀(PO₄)₆O (COXA);
calcium carbonate apatite Ca₁₀(PO₄)₆CO₃ (CCA); and
calcium carbonate hydroxyapatites, e.g., Ca₁₀(PO₄)₅(OH)(CO₃)₂ and
Ca₁₀(PO₄)₄(OH)₂(CO₃)₃ (CCHA).

Calcium phosphates useful herein also include calcium-deficient calcium phosphates in which the molar or mass ratio of Ca:P is reduced by about 20% or less, preferably about 15% or less, preferably about 10% or less, relative to the corresponding calcium non-deficient species, examples of which include calcium-deficient hydroxyapatites, e.g., Ca_{10-X}(HPO₄)_{X}(PO₄)_{6-X}(OH)_{2-X} (0≦X≦1) (CDHA); calcium-deficient carbonate hydroxyapatites (CDCHA); calcium-deficient carbonate apatites (CDCA); and other calcium phosphate compounds and salts known as useful in the bone graft material field, e.g., calcium polyphosphates; and calcium-, phosphate-, and/or hydroxyl-“replaced” calcium phosphates, as further described below.

Calcium-replaced calcium phosphates are also useful herein, including homologs of any of the above in which some of, preferably a minority of (preferably about or less than: 40%, 35%, 33.3%, 30%, 25%, 20%, 15%, or 10% of) the calciums are substituted with monovalent and/or divalent metal cation(s), e.g., sodium calcium homologs thereof, such as CaNa(PO₄).

Phosphate-replaced calcium phosphates are also useful herein, including homologs of any of the above in which some of, preferably a minority of (preferably about or less than: 40%, 35%, 33.3%, 30%, 25%, 20%, 15%, or 10% of) the phosphate groups are substituted with carbonate, hydrogen phosphate, and/or silicate groups.

Hydroxyl-replaced calcium phosphates are also useful herein, including homologs of any of the above hydroxyl-containing materials in which some of, preferably a minority of (preferably about or less than: 40%, 35%, 33.3%, 30%, 25%, 20%, 15%, or 10% of) the hydroxyl groups are substituted with F, Cl, I, or CO₃.

In some embodiments of a calcium-replaced homolog, the monovalent metal cation can be an alkali metal cation, for example, sodium; or it can be Cu(I); or a combination thereof. In some embodiments of a calcium-replaced homolog, the divalent metal cation can be an alkaline earth metal, including beryllium, magnesium, strontium, barium, and combinations thereof. In some embodiments of a calcium-replaced homolog, the divalent metal cation can be a divalent transition metal, including chromium, cobalt, copper, manganese, zinc, and combinations thereof.

In some embodiments of a hydroxyl-replaced homolog, the halide can be fluoride, chloride, or iodide. Examples of such hydroxyl-replaced homologs include calcium haloapatites, calcium haloahydroxypatites, and calcium halo-oxyapatites, the latter having a formula of, e.g., Ca₁₅(PO₄)₉(X)O wherein X is F, Cl, or I.

In various embodiments, the calcium phosphate ceramic comprises β-tricalcium phosphate and/or amorphous calcium phosphate. For the preparation of the calcium phosphate ceramic, the powdered mixture can be sterilized by autoclaving, irradiated with ionizing irradiation, or chemically treated. When the growth conditions for bone forming cells such as osteoblasts or osteoblast progenitors cells like mesenchymal stem cells are taken into account, the calcium phosphate ceramic can be adjusted to a pH range from about 7.0 to about 7.4.

In some embodiments, one of three common calcium phosphate ceramics can be used: hydroxyapatite ceramics (Ca₅(PO₄)₃OH, HAp); β-tricalcium phosphate ceramics (Ca₃(PO₄)₂, β-TCP) which are more soluble than HAp in physiological conditions; and biphasic calcium phosphate ceramics, comprising mixtures of HAp and β-TCP which exhibit intermediate resorbability depending on the exact composition of the mixture. Mixtures of one or more of the three calcium phosphate ceramics can also be used.

In various embodiments, calcium phosphate ceramics include tetracalcium phosphate monoxide (Ca₄O(PO₄)₂,); dicalcium phosphate (CaHPO₄, or CaHPO₄.2H₂O), e.g. the BoneSource® HAp cement; α-tricalcium phosphate; monocalcium phosphate monohydrate (Ca(HPO₄)₂. H₂O; calcium carbonate (Norian SRS® cement); ceramics based on alpha tricalcium phosphate; dicalcium phosphate and calcium carbonate mixtures; and ceramics based on mixtures of beta-tricalcium phosphate and mono-calcium phosphate. In various embodiments, calcium phosphate ceramics can comprise beta-tricalcium phosphate having a porosity of not less than 30%, 40%, 50%, 60%, 70%, 80% and not less than 90%. In some embodiments, calcium phosphate ceramics can comprise ultraporous beta-tricalcium phosphate.

In various embodiments, mixtures of calcium phosphates and other calcium salts can be incorporated into the formulations of the present teachings. In some embodiments, the composition can additionally comprise calcium sulfate salts, calcium carbonates, calcium apatites, porous coralline ceramics, bioactive glass comprising calcium oxide, apatite/wollastonite glass ceramics, calcium silicates, resorbable polymers such as polylactic acid, polysulfones, polyolefins, polyvinyl alcohol, polyalkenoics, polyesters, polyglycolic acid, polysaccharides, polyglycolates, polycaprolactone, and mixtures thereof.

Without limiting the mechanism, function or utility of the present invention, the biological behavior of calcium phosphate ceramics can depend on the physical and chemical properties of the calcium phosphate ceramic, and specifically on their Ca/P atomic ratio. In some embodiments, the bone graft composites of the present teachings can also comprise pores of various sizes within the graft material that promote the in-growth of new bone cells. Preferably the pore size of the ceramic material is from about 1 to about 1000 microns, preferably from about 40 to about 750 microns. Preferably the Ca/P atomic ratio is from about 0.5 to about 2.0, preferably from about 1.4 to about 1.6.

In various embodiments the amount of calcium phosphate ceramic can be incorporated into the graft at a level of less than about 40%, less than 30%, less than 20%, less than 15%, less than 10%, less than 8%, less than 5%, less than 3%, or more than 1%, more than 3%, more than 5%, more than 7%, more than 10% or more than about 15%.

In some embodiments, calcium phosphate ceramics can be provided in pellet, or granule, or powdered form, or in combinations thereof. The calcium phosphate can be obtained from several commercial sources including Kensey Nash Corporation, (Exton, Pa. USA). The calcium phosphate ceramics described herein can be used in bone graft composites having various product forms, including injectable or moldable pastes or moldable putties for temporary bone filling, pre-hardened shaped graft implants, and coatings for orthopedic devices and prostheses.

### Demineralized Bone (DBM)

The compositions of the present invention comprise demineralized bone matrix. The term “demineralized” as used herein refers to bone or bone material containing less than its original mineral content and is intended to encompass “substantially demineralized,” “partially demineralized,” and “completely demineralized” bone material. In various embodiments, the calcium content in the demineralized bone matrix can be less than about two percent. The demineralized bone matrix can, in various embodiments, comprise osteoinductive factors, including bone morphogenetic proteins, insulin-like growth factor-1 (IGF-1), fibroblast growth factor (FGF) and transforming growth factor-beta1 (TGF-beta1), osteogenin, osteonectin, and osteocalcin. In various embodiments, bone graft composites of the present teachings containing DBM can enhance bone growth when the graft further comprises progenitor stem cells such as autologous or allogeneic mesenchymal stem cells. As used herein, the terms “autologous” and “autogenous” are synonymous and refer to involving one individual as both donor and recipient.

Demineralized bone matrix can be produced by acid extraction, thermal freezing, irradiation, or physical extraction of inorganic minerals from human or animal bone. The moisture level of the demineralized bone matrix is preferably controlled. This may be accomplished in a number of ways. For example, the demineralized bone matrix can be air-dried or freeze-dried. Air dried demineralized bone matrix can include greater than about 10 weight percent of moisture, while in certain circumstances, freeze dried demineralized bone matrix can include less than about 6 weight percent of moisture. The demineralized bone matrix preferably includes between about 5 and about 30 weight percent (e.g., between about 5-20 weight percent, between about 10-15 weight percent, or between about 10-12 weight percent, or between about 5-10 weight percent) of moisture, e.g., water. In various embodiments, the demineralized bone matrix includes greater than or equal to about 6, 10, 12, 14, 16, 18, 20, 22, 24, 26, or 28 weight percent of moisture; and/or less than or equal to about 30, 28, 26, 24, 22, 20, 18, 16, 14, 12, or 6 weight percent of moisture. In some embodiments, the bone used to manufacture the demineralized bone matrix can be cortical, cancellous, cortico-cancellous of autogenous, allogeneic, xenogeneic or transgeneic in origin.

In various embodiments of the present teachings, demineralized bone matrix can be supplied as powdered cortical or cancellous bone or dry chips ranging in size from about 10 μm to about 10 mm, from about 50 μm to about 5 mm, from about 100 μm to about 1 mm, from about 150 μm to about 0.8 mm, or from about 200 μm to about 0.75 mm.

In certain embodiments, the demineralized bone matrix, along with other materials in the bone graft composite, are packaged in a kit and subjected to sterilization, e.g., electron beam sterilization, prior to being used. In addition or alternatively, the demineralized bone matrix can be packaged separately from the other powdered ingredients (e.g., collagen, calcium phosphate ceramic and bioactive factors). Demineralized bone matrix is commercially available, e.g., from Lifelink Tissue Bank (Tampa, Fla. USA), Community Tissue Services (Dayton, Ohio USA), Allosource (Denver, Colo. USA.) or DCI Donor Services (Nashville, Tenn. USA).

In some embodiments, the demineralized bone matrix has a particle size of about 50-850 microns, e.g., about 110-710 microns. The particle size can be greater than or equal to about 50, 150, 200, 250, 300, 350, 400, 450, 500, 550, 600, 650, 700, 750, or 800 microns; and/or less than or equal to about 850, 800, 750, 700, 650, 600, 550, 500, 450, 400, 350, 300, 250, 200, 150, or 50 microns.

In various embodiments the amount of demineralized bone matrix is incorporated into the graft at a level of less than about 90%, less than 85%, less than 80%, less than 75%, less than 70%, less than 65%, less than 60%, less than 55%, or more than 50%, more than 55%, more than 60%, more than 65%, more than 70% or more than about 75%.

### Collagen

The compositions of the present invention comprise a collagen material. Collagen's basic structure consists of three polypeptide chains, each with a repeating primary amino acid sequence of -glycine-X—Y—. In various embodiments, the collagen may be in a polymerized fibrous form that has a long three-dimensional architecture with multiple cross-links. In various embodiments, the collagen component can be fibrillar collagen, atelopeptide collagen, telopeptide collagen or tropocollagen and can be collected from a variety of animal sources, including human. In some embodiments, the collagen is a mammalian collagen. In some embodiments, the collagen can be human collagen. In some embodiments, the collagen is selected from the group comprising human Type I, II, III or IV, bovine Type I collagen, and porcine Type I collagen. In some embodiments, the collagen carrier can be purified fibrillar bovine tendon Type I collagen. In various embodiments, the amount of collagen present in the materials and compositions of the present invention is preferably from about 10% by weight to about 45% by weight, from about 15% by weight to about 40% by weight, or from about 20% by weight to about 35% by weight.

In various embodiments, the admixture of the collagen carrier with the calcium phosphate ceramic results in a graft that is highly porous with a broad pore size distribution. Without limiting the function, mechanism or utility of the present invention, collagen may support the growth and differentiation of bone forming progenitor stem cells, particularly those stem cells that differentiate into osteoblast like cells expressing osteonectin, osteopontin and CD44. Furthermore, the collagen may improve the resorption profile of bone-grafted tissue enhancing the remodeling of synthetic bone over to natural bone.

In various embodiments, the bone graft composites comprising collagen carriers can have augmented properties, and improved moldability over the same bone graft composition without the collagen carrier present. The resorption profile of some of the embodiments of the present teachings can vary depending upon the amount, nature, and source of the collagen or other components used. In some embodiments according to the present teachings, within about 4-6 weeks, about 85%-95% of the collagen carrier within the bone graft in vivo will have been resorbed.

### Bioinductive Cellular Solutions

The compositions of the present invention comprise a bioinductive cellular solution. As described herein, the phrase “bioinductive cellular solution(s)” refers to hydrating solutions, suspensions, or other fluids that contain cells that are capable of differentiating into bone producing and/or bone remodeling cells. In one of many examples, mesenchymal stem cells can be illustrative of a cell type that can be differentiated into bone forming or bone remodeling cells. The source of the bioinductive cellular solutions can be autologous, allogeneic, xenogeneic or transgeneic. Examples of bioinductive cellular solutions can include blood, blood components, bone marrow aspirate and adipose tissue liposuction aspirate. In some embodiments, the bioinductive cellular solution can be autologous bone marrow aspirate. In some embodiments the blood and aspirated bone marrow or adipose tissue liposuction aspirate can be collected during routine medical visits (preoperatively), or collected intraoperatively during the bone graft or orthopedic prosthesis implantation. Without limiting the function, mechanism or utility of the present invention, the bioinductive cellular solution may function as either or both of a hydrating solvent for the bone graft composite and as a source of inducible and determined osteoprogenitor cells.

In some embodiments the patient can have a sample of blood withdrawn before or during surgery to hydrate the powdered bone graft precursor comprising powdered demineralized bone matrix, collagen carrier and calcium phosphate ceramic. In some embodiments the blood can be further treated with an anticoagulant including heparin, sodium citrate and EDTA. In some embodiments, the patient's blood can be further stabilized with physiological buffers including Hank's Balanced Salt Solution, phosphate buffered saline or tissue culture medium designed to support hematopoietic cells (for example minimal essential medium).

In various embodiments, bone marrow and/or adipose tissue liposuction aspirate can be used as the bioinductive cellular solution. Autologous or allogeneic bone marrow or adipose tissue liposuction aspirate can be collected from the donor prior to surgery, or specifically for implantation of a bone graft or bone graft coated prosthesis intraoperatively. For bone marrow removal, the skin over the iliac crest of the pelvic bone and the outer surface of the bone itself can be numbed with local anesthesia by injection or intra-venous application. Then, a larger needle can be inserted into the iliac crest and marrow is drawn into a syringe. Marrow cells can be suctioned two to six times during a 15-minute procedure. In some embodiments, the aspirated bone marrow contains peripheral blood. The bone marrow aspirate can in some embodiments be anti-coagulated. Suitable anti-coagulants can include, for example, heparin, sodium citrate, and EDTA. In some embodiments, the bone marrow aspirate can be further stabilized with physiological buffers including Hank's Balanced Salt Solution, phosphate buffered saline, tissue culture medium designed to support hematopoietic cells (for example minimal essential medium).

The collection of adipose tissue liposuction aspirate can be collected in any medically approved procedure known in the art. Essentially, the adipose tissue is disrupted with some input energy and one or more injections of buffer or saline can be injected into the adipose tissue to facilitate liposuction removal of adipose tissue from the patient. Removal and purification of adipose tissue stem cells is described in U.S. Patent Publication No. 2006/0051865 and is hereby incorporated in its entirety.

In some embodiments, the bone marrow or adipose tissue liposuction aspirate can be concentrated to obtain a concentrated pool of bone progenitor cells by centrifuging the bone marrow or adipose tissue liposuction aspirate at 400 times gravity for about ten minutes.

The volume of bioinductive cellular solution varies depending on the desired consistency of the bone graft composition. In some embodiments, the volume of bioinductive cellular solution added to the powdered bone graft precursor can affect the time the composition takes to set, i.e., the set time.

In various embodiments, the bioinductive cellular solution can optionally include, for example, biological factors or “bioactive factors.” Bioactive factors can be any natural, recombinant or synthetic factor that promotes the growth of bone directly or indirectly and can facilitate or be implicated in normal bone remodeling. In some embodiments, bioactive factors can include: bone growth factors, extracellular matrix proteins, hormones, cytokines, cell signaling proteins, platelet concentrate, blood, pharmaceutical actives, or combinations of these materials. Examples of bone growth factors include transforming growth factor-beta (TGF-β) including the five different subtypes (TGFβ1-5); bone morphogenetic factors (BMPs); platelet-derived growth factors (PDGFs); insulin-like growth factors (e.g., IGF I and II); and fibroblast growth factors (FGFs). Examples of pharmaceutical actives include antibiotics, chemotherapeutic agents, and analgesics. Examples of antibiotics include sulfonamides, furans, macrolides, quinolones, tetracyclines, vancomycin, cephalosporins, rifampins, and aminoglycosides such as tobramycin and gentamicin. In some embodiments, the active is a combination of a tetracycline and a rifampin. Examples of chemotherapeutic agents include cis-platinum, ifosfamide, methotrexate, and doxorubicin hydrochloride (Adriamycin®). Examples of analgesics include anesthetics such as lidocaine hydrochloride (Xylocaine®), bipivacaine hydrochloride (Marcaine®), and non-steroidal anti-inflammatory drugs such as flurbiprofen, diclofenac, sulindac, oxaprozin, diflunisal, piroxicam, ibuprofen, indomethacin, ketoprofen, etodolac, meclofenamate sodium, meloxicam, fenoprofen, naproxen, mefanamic acid, nabumetone, tolmetin sodium, COX-2 Inhibitors (such as celecoxib, rofecoxib, and valdecoxib), and ketorolac. Bioactive factors can be titrated to obtain optimal biological activity and proper handling properties of the bone graft.

In various embodiments, the present invention provides bone graft composites that are optimized in terms of one or more of composition, bioactivity, porosity, pore size, protein binding potential, degradability or strength for use in both load bearing and non load bearing bone grafting applications. Preferably, bone graft materials are formulated so that they promote one or more of three processes involved in bone healing which can occur with the application of a single graft material: osteogenesis, osteoinduction, and osteoconduction. Osteogenesis is the formation of new bone by the cells contained within the graft. Osteoinduction is a chemical process in which molecules contained within the graft (for example, bone morphogenetic proteins and TGF-β) convert the patient's or other bone progenitor cells into cells that are capable of forming bone. Osteoconduction is a physical effect by which the matrix of the graft forms a scaffold on which bone forming cells in the recipient are able to form new bone.

Without limiting the function, mechanism or utility of the present invention, the bone graft material can, in some embodiments, provide conditions favorable for osteogenesis. Osteogenesis primarily involves two types of bone formation. The first is intramembranous bone formation, the second is endochondral bone formation. The difference between the two osteogenic processes revolves around the use of cartilage as the starting material for bone. Bone graft composites described herein can be used to facilitate the replacement and filling of bone material in and around preexisting host bone. In some embodiments, the grafts described herein can also be used to produce cartilage which is then mineralized to form bone. In some instances, mesenchymal stem cells present in the bone graft or at the site of implantation can differentiate into chondrocytes first, followed by deposition of extra cellular matrix and invasion of blood vessels and other bone forming cells. Thus, such bone graft composites can interact intimately with the surrounding bone tissue and blood vessels. Such bone graft composites are preferably biocompatible, i.e. the graft materials are not inflammatory and are conducive to cellular in-growth and differentiation of progenitor bone cells within the bone graft.

Furthermore, without being bound to theory, in some embodiments the bone graft composites of the present teachings allow the host's circulating mesenchymal stem cells and the graft's embedded stem cells to produce new bone at the treated site (osteoinduction). Osteoinductive factors (e.g. BMPs, and other growth factors) contained within the bone graft composite can attract circulating and embedded mesenchymal stem cells to the site of repair and provide the necessary differentiation signals to coordinate the differentiation of mesenchymal stem cells into bone forming and remodeling cells.

The bone graft composites of the present teachings can provide an osteoconductive scaffold comprising calcium phosphate ceramics. Without being bound to theory, the calcium phosphate ceramics along with collagen carriers can provide an osteoconductive framework for the implanted progenitor cells and local osteocytes to differentiate into bone forming cells and deposit new bone. The use of calcium phosphate ceramics in the present teachings can provide for a slow degradation of the ceramic, which results in a local source of calcium and phosphate for bone formation. Therefore, new bone can be formed without calcium and phosphate loss from the host bone surrounding the defect site. This avoids fusion at the expense of reduced bone mineral density of adjacent host bone. The bone graft composite described herein can provide pores of various sizes for bone formation to occur and also provides a scaffold for bone in-growth.

### Methods of Making Bone Graft Materials

The composition can be formed by providing the powdered bone graft precursor (e.g., calcium phosphate ceramic, demineralized bone matrix, and collagen carrier) and contacting, e.g., mixing, the powdered components with the bioinductive cellular solution to form the bone graft composite. The bone graft composite can be implanted in a patient in a paste or putty form (i.e., as a hydrated precursor). In some embodiments, the bone graft composite is non-setting, thus the likelihood that the material will “set up” prior to application to the surgical site and become unusable is minimized. In some embodiments, this feature is particularly useful in the surgical setting, where custom manipulation of the moldable bone graft into the void site or placement into a particular device is typically required.

Alternatively, the inventive bone graft implant can be pre-hardened outside the body and implanted at a later time after wicking with the patient's blood or bone marrow and/or adipose tissue liposuction aspirate. This approach is useful in various applications where custom shapes are not essential, and where production of large numbers of implants is desired. Accordingly, in various embodiments, the bone graft composites of the present teachings can be prepared ex vivo in a variety of shapes and forms and introduced into the patient at the implant site using methods appropriate to the form of the implant and nature of the malady. Such shapes include, for example, a cylinder, wedge, plate, threaded cylinder, fibular wedge, femoral strut or tibial strut, or any solid free-form fabrication structure. In some embodiments, the composition is capable of bearing loads upon implantation. In some embodiments, certain orthopedic devices, e.g. fusion devices, are coated with the bone graft composition prior to placement in the patient's body.

In some embodiments, the bone graft composites can be prepared as an injectable paste. In various embodiments, a bioinductive cellular solution can be added to one or more powdered bone graft precursors to form an injectable hydrated bone graft paste. The precise amount of bioinductive cellular solution will vary depending on the desired consistency of the paste and the nature of the powdered bone graft precursor used to prepare bone graft composite. The paste can be injected into the implant site, preferably using a twelve to eighteen-gauge needle syringe. In some embodiments, the bone graft paste can be prepared prior to implantation and/or store the paste in the syringe at sub-ambient temperatures until needed. In some embodiments, injection by syringe into a body cavity or intermedullary space can be aided by the use of vacuum to aid in displacing fluids or gases. In some embodiments, application of the bone graft composite by injection can resemble a bone cement that can be used to join and hold bone fragments in place or to improve adhesion of, for example, a hip prosthesis. Implantation in a non-open surgical setting can also be performed.

In various embodiments, the bone graft composites of the present teachings can be prepared as formable putty. A bioinductive cellular solution can be added to one or more powdered bone graft precursors to form a putty-like hydrated bone graft composite. The precise amount of bioinductive cellular solution will vary dependent upon the desired consistency of the putty and the nature of the powdered bone graft precursor used to prepare the bone graft composite material. The hydrated bone graft putty can be prepared and molded to approximate any implant shape. The putty can then be pressed into place to fill a void in the bone, tooth socket or other site. In some embodiments, bone graft putty can be used to repair bone defects in non-union bone or in other situations where the fracture, hole or void to be filled is large and requires a degree of mechanical integrity in the implant material to both fill the gap and retain its shape.

The present invention provides methods of treating a bone defect in a human or other animal subject, comprising applying to the site of the defect a composition comprising: calcium phosphate; demineralized bone matrix; collagen; and bioinductive cellular solution. As referred to herein such “bone defects” include any condition involving skeletal tissue which is inadequate for physiological or cosmetic purposes. Such defects include those that are congenital, the result from disease or trauma, and consequent to surgical or other medical procedures. Such defects include for example, a bone defect resulting from injury, defect brought about during the course of surgery, osteoporosis, infection, malignancy, developmental malformation, and bone breakages such as simple, compound, transverse, pathological, avulsion, greenstick and communuted fractures. In some embodiments, a bone defect is a void in the bone that requires filling with a bone graft composite of the present teachings.

In various embodiments, the bone graft compositions can be first lyophilized and then rehydrated upon implantation of the bone graft with the patient's blood, bone marrow aspirate, adipose tissue liposuction aspirate or combinations thereof. In various embodiments, the bone graft composites are made prior to the time of implantation and are shaped to fit into a particular bone void or a specific device.

In some embodiments, the bone graft composites can also be used in the form of pre-shaped blocks hardened ex vivo. In some embodiments, the bone graft composites prepared according to the present teachings are combined with setting agents such as organic acids for example, citric acid and optionally sodium phosphate, which allow the bone graft composite to desiccate and harden. In some embodiments, pre-hardened bone graft composites can be formulated containing greater quantities of calcium phosphate ceramic than the bone graft composites resembling paste or putty materials to ensure proper hardening of the bone graft composition. The calcium phosphate ceramics described in the present teachings can be admixed with other powdered components such as demineralized bone matrix, collagen carriers and other bioactive factors and hydrated with bioinductive cellular solutions either before the bone grafting surgery or intraoperatively.

In some embodiments, dry powdered bone graft precursor can be applied directly to a bone defect. Hydration and conversion of the powdered bone graft precursor into the bone graft composite material can occur at the bone defect site by direct exposure to blood or injected with bone marrow and/or adipose tissue liposuction aspirate in situ. Such application can be used when for example; the bone defect is accompanied by excessive bleeding. The hydroscopic nature of the powdered bone graft precursor can, in some embodiments, serve one or more functions of absorbing body fluids containing osteoprogenitor cells, providing a physical barrier to protect the wound site, and providing a bone graft composite with determined and inducible osteogenic progenitor cells at the defect site.

In some embodiments of the present teachings, the bone graft composites can be prepared and processed into a pre-hardened graft composite having any predetermined shape. This can be accomplished by preparing a hydrated settable bone graft composite as described above, injecting or pressing the hydrated precursor bone graft composite into a mold, and allowing the precursor bone graft composite material to desiccate and harden into a predetermined finished bone graft article. Alternatively, the bone graft composite can be prepared as a solid block or other such geometry and shaped into the desired object using drills or other such shaping tools known in the art. Shaped bone graft composites can be used in the production of resorbable objects such as anchors for tooth implants, spacers for cervical fusion, resorbable screws and plates, and slowly resorbable shapes for augmentation. When the resorbable article or device is ready for implantation, the device or article can be soaked in the patient's blood, bone marrow aspirate, or adipose tissue liposuction aspirate and positioned into place.

Bone graft composites described herein can be used to join two or more bone pieces together and/or to improve healing of bone fractures by filling a gap left by a fracture or a space caused by compressive damage as a result of the fracture. In some embodiments, the bone graft composites can be used to stabilize non-union bone fractures because the implant is osteogenic and can fill the void with newly synthesized bone in vivo. The bone graft implant of the present teachings can be used to fill the bone void without open surgery. In some embodiments, the bone defect site can be guided by x-ray to ensure proper positioning of the injection needle. The bone graft implant can then be directly injected into the defect site. X-ray or MRI visualization can be used, if desired, to confirm placement. In some embodiments, when the gap is particularly large, the bone graft composite can immobilize or “fix” the defect and then fill the gap with newly synthesized bone.

In various embodiments, the bone graft composites described herein can be used to repair and heal bony defects including structurally compromised bone, for example, as a result of trauma or infection and bone having voids and/or fractures. It will be appreciated that bone healing applications involving bone graft composites of the present teachings include, but are not limited to, filling interbody fusion devices/cages (ring cages, cylindrical cages), placement adjacent to cages (i.e., in front cages), placement in the posterolateral gutters in posterolateral fusion (PLF) procedures, backfilling the iliac crest, acetabular reconstruction and revision hips and knees, large tumor voids, use in high tibial osteotomy, burr hole filling, and use in other cranial defects. The bone graft composite material can be adapted for use in PLF by placement in the posterolateral gutters, and in onlay fusion grafting. Additional uses can include craniofacial and trauma procedures that require covering or wrapping of the injured/void site with the bone graft composites described herein. In some embodiments, the bone graft composite material can be fashioned into cylinders which can be used to fill spinal cages and large bone voids, and for placement along the posterolateral gutters in the spine.

In some embodiments, infected or otherwise damaged bone tissue may require removal prior to filling the defect with bone graft compositions of the present teachings. The techniques required to aseptically remove infected and/or damaged bone are well known in the orthopedic surgical fields. The bone graft compositions described herein can be used to replace bone tissue removed due to infection and or trauma.

In some embodiments, where the bone has been crushed or fragmented, the bone fragments can be “glued” together in its physiological state. In some embodiments, the bone graft composites of the present teachings can be used to hold the repaired bone fragments in place while the natural bone matrix regrows and replaces the “glue” that holds the fragment(s) together.

The bone graft composites can also be used to heal compression fractions, such as compression of the tibia. The cortical bone surface can be re-aligned and fixed in place using mechanical fixation and the bone graft composites of the present teachings can be used to fill the void created by the compressive destruction of the bone.

In some embodiments, the bone graft composite can be used to secure pins, screws and other more complicated orthopedic devices that are used to fix bone in place. By immobilizing the fracture using orthopedic hardware and embedding the hardware in bone graft paste, potential voids are filled, thereby expediting new bone formation around the immobilizing or orthopedic device (e.g. a bone screw). In some embodiments, the bone graft composite acts to distribute the force imparted by the screw across a greater surface area, thereby reducing the likelihood of pull out or early bone resorption.

In some embodiments, the bone graft composites of the present teachings can be used in arthroplasty procedures of the hip, knee, shoulder and other joints to fix plastic and metal prosthetic parts to living bone. In some embodiments, this approach can be effectively employed in repair of broken hipbones, where a hip prosthesis can be used to reinforce the weight-bearing femoral neck of the femur.

In some embodiments where minimal surgical intervention is required (for example, to repair a fracture), the bone graft composites can be a paste and introduced by syringe into the bone defect. In some embodiments, bone defects are larger than a fracture and require substantial intervention, i.e., during open surgery, the bone graft composite can be used as moldable putty. In some embodiments, the improved handling properties of the putty can provide the physician increased control over the final shape of the implanted device and improves the bone graft's ability to support and function the neighboring bone.

In various embodiments, the bone graft material can be hardened ex vivo and can be used for intraoperative support of hardware, such as orthopedic hardware, e.g., bone plates, distal radius hardware, and hardware used for tibial plateau fractures. Prior to implantation into or around the surface of the orthopedic device, the hardened bone graft composites can be mixed with a bioinductive cellular solution wherein the hardened bone graft material readily wicks up blood and bone marrow aspirate prior to implantation. In some embodiments, the resulting hardened bone graft can be shaped using any conventional shaping or grinding technique. In various embodiments, the hardened and shaped bone graft composites are combined and hydrated with a bioinductive cellular solution.

In some embodiments, the bone graft composites can be hydrated into a paste and molded into any particular shape using any commonly available molding technique. In some embodiments, wedge shaped bone grafts can be used in high tibial osteotomies and other geometries can find further utility in other bone defect repairs. When the bone graft composite is to be placed into a fusion device, cage, plate etc, the bone graft composite can be hydrated or rewetted with the patient's blood, bone marrow and/or adipose tissue liposuction aspirate intraoperatively.

The present technology is further illustrated through the following non-limiting examples.

### EXAMPLES

### Example 1

A mixture of powders (6.3 grams) is prepared comprising 0.3 grams of type I collagen or mixtures of animal or human collagen, 2.00-2.67 grams of demineralized bone matrix (LifeLink Tissue Bank, Tampa, Fla. USA), having a density of 0.33 g/cc, 100-1000 μm particle size; 3.5-4.5 grams of beta-tricalcium phosphate 50-400 μm particle size (Kensey Nash Corp. Exton, Pa. USA). The powders are mixed and sterilized. The sterile mixture of powders is then lyophilized and hydrated with bone marrow aspirate to form a continuous non-moldable, non-flowable scaffold. The graft material is shaped into an elliptical wedge, suitable for repairing defects of a high tibial osteotomy. The elliptical wedge has a major axis of about 7 cm and a minor axis of about 2 cm. When expanded, the elliptical wedge has a major axis of 7 cm and a minor axis of 5 cm. The wedge has a height on the tapered distal edge to 5 mm and the large proximal edge can be 25 mm.

In the above example, the powders are hydrated with blood and with adipose tissue liposuction aspirate, with substantially similar results. Also in the above example, a second shape is found as an elliptical cylinder format that can be fit into a metal fixation device. In this embodiment, the cylinder can have a major axis of about 14.5 mm and a minor axis of about 5 mm. The height of the bone graft before hydration can be around 6 mm and the height of the graft after hydration can be around 25 mm.

### Example 2

A bone paste or putty is manufactured by combining 60-80% (vol. %) of 100-1000 μm demineralized bone matrix (LifeLink Tissue Bank, Tampa, Fla. USA), −3-5% (vol. %) of collagen carrier Type I collagen (Kensey Nash Corp, Exton, Pa. USA); and 3-5 g of beta-tricalcium phosphate (IsoTis OrthoBiologics, Irvine, Calif. USA). The powdered bone graft material having a moisture content of less than 5% is lyophilized and then rehydrated using blood by mixing together the powders and the solution to form a composition having a putty-like consistency. The putty-like bone graft material is manually manipulated to be inserted into a bone void or defect.

In the above example, the powdered bone graft material is hydrated with a greater volume of blood than that used to make the putty graft material, to form a viscous liquid paste that is loaded into a syringe and administered in volumes of 5 cc, 10 cc and 15 cc using EBI Graft Delivery Syringes (GDS syringes) for injection into bone voids or as coating materials for various orthopedic appliances and devices in which bone formation is required between the inserted article and the preexisting bone into which the article is inserted for example spine fixation appliances and devices.

The examples and other embodiments described herein are exemplary and not intended to be limiting in describing the full scope of compositions and methods of this invention. Equivalent changes, modifications and variations of specific embodiments, materials, compositions and methods may be made with substantially similar results.

What is claimed is:

1. A bone graft composition, comprising:
demineralized bone matrix;
calcium phosphate;
collagen; and
bioinductive cellular solution.

2. A composition according to claim 1, wherein the demineralized bone matrix is demineralized, partially demineralized or superficially demineralized cortical, cancellous or cortico-cancellous bone.

3. The composition of claim 2, wherein the demineralized bone matrix is selected from the group consisting of bone chips, bone particles, bone morsels, ground bone, bone powders, and mixtures thereof.

4. A composition according to claim 1, wherein the calcium phosphate is selected from the group consisting of tricalcium phosphate, hydroxyapatite, tetra calcium phosphate, amorphous calcium phosphate, and mixtures thereof.

5. A composition according to claim 1, wherein the collagen carrier is selected from the group consisting of mammalian type I, type, II, type III, type IV, type IX, type X, type XI and type XII collagen, and mixtures thereof.

6. A composition according to claim 5, wherein, the collagen carrier is selected from the group consisting of mammalian type I, type II, type III, type IV collagens, and mixtures thereof.

7. A composition according to claim 1, wherein, the bioinductive cellular solution is selected from the group consisting of bone marrow aspirate, blood, adipose tissue liposuction aspirate, cultured cells, progenitor cells, stem cells, stromal cells, and mixtures thereof.

8. A composition according to claim 7, wherein the bioinductive cellular solution comprises autologous bone marrow aspirate.

9. A composition according to claim 7, wherein the bioconductive cellular solution comprises blood selected from the group consisting of autologous blood, autologous buffy coat cells, autologous plasma, allogeneic blood, allogeneic buffy coat cells, allogeneic plasma, and mixtures thereof.

10. A composition according to claim 7, wherein the bioconductive cellular solution comprises mesenchymal stem cells.

11. A composition according to claim 7, wherein the bioinductive cellular solution comprises one or more of physiological saline, physiological buffers and water.

12. A composition according to claim 1, comprising:
from about 55 to about 80 parts of demineralized bone matrix;
from about 1 to about 10 parts of calcium phosphate;
from about 15 to about 40 parts of collagen; and
from about 1 to about 5 parts of a bioinductive cellular solution.

13. A composition according to claim 1, comprising one or more bioactive factors selected from the group consisting of growth factors, structural proteins, cytokines, antibiotics, chemotherapeutics, serum proteins, and mixtures thereof.

14. A method for treating a bone defect site, said method comprising applying to the site of said defect a bone graft composition comprising demineralized bone matrix, calcium phosphate ceramic, collagen, and a bioinductive cellular solution.

15. A method according to claim 14, wherein said composition is admixed intraoperatively and implanted into said sites where defective osseous tissue has been removed from said site.

16. A method according to claim 15, wherein said composition is effective to induce bone formation in said defective osseous tissue.

17. A method according to claim 14, wherein said composition is implanted surgically into the site.

18. An implant comprising a composition of claim 1, possessing the shape of a cylinder, wedge, plate, threaded cylinder, fibular wedge, femoral strut or tibial strut, said implant being capable of initially bearing loads upon implantation.

19. An implant according to claim 18, wherein the shaped composition is hardened ex vivo and implanted surgically.

20. An implant according to claim 18, wherein the shaped composition is infused with a bioinductive cellular solution prior to implantation into the patient.

21. A method for implanting to a patient an orthopedic device having contact on at least one surface with bone, comprising:
applying to at least one surface of the orthopedic device a bone graft composition comprising demineralized bone matrix, calcium phosphate, collagen, and bioinductive cellular solution; and
implanting the orthopedic device into the patient.

22. A method according to claim 21, wherein the orthopedic device is adopted for use in arthroplasty, orthopedic distraction, or fixation, wherein the orthopedic device is to remain in permanent contact with the supporting bone.

23. A method of preparing a bone graft material comprising:
a) providing a matrix comprising:
demineralized bone;
calcium phosphate; and
collagen; and
b) mixing said matrix with a bioinductive cellular solution to yield a bone graft material containing bioinductive cells distributed within the bone graft material.

24. A method according to claim 23, wherein the demineralized bone matrix is demineralized, partially demineralized or superficially demineralized cortical, cancellous or cortico-cancellous bone.

25. A method according to claim 23, wherein the demineralized bone matrix is selected from the group consisting of bone chips, bone particles, bone morsels, ground bone and bone powders, and mixtures thereof.

26. A method according to claim 23, wherein the calcium phosphate is selected from the group consisting of tricalcium phosphate, hydroxyapatite, tetra calcium phosphate, poorly crystalline apatite and mixtures thereof.

27. A method according to claim 23, wherein the collagen carrier is selected from the group consisting of mammalian type I, type II, type III, type IV collagen, and mixtures thereof.

28. A method according to claim 23, wherein, the bioinductive cellular solution is selected from the group consisting of blood, bone marrow aspirate, adipose tissue liposuction aspirate, allogeneic stem cells, cultured human and/or animal stem and stromal cells, and mixtures thereof.

29. A method according to claim 28, wherein the bioinductive cellular solution comprises autologous bone marrow aspirate.

30. A method according to claim 23, wherein the bioinductive cells comprise mesenchymal stem cells.

31. A method according to claim 28, wherein the bioconductive cellular solution comprises blood selected from the group consisting of autologous blood, autologous buffy coat cells, autologous plasma, allogeneic blood, allogeneic buffy coat cells, allogeneic plasma, and mixtures thereof.

32. A method according to claim 28, wherein the bioconductive cellular solution comprises mesenchymal stem cells.

33. A method according to claim 28, wherein the bioinductive cellular solution comprises one or more of physiological saline, physiological buffers and water.

34. A kit, comprising:
a mixture comprising:
demineralized bone matrix;
calcium phosphate; and
collagen; and
instructions for preparing said mixture by admixture with bioactinductive cellular solution.

35. A kit according to claim 34, wherein the bioinductive cellular solution is selected from the group consisting of autologous bone marrow aspirate, autologous blood, autologous buffy coat cells, autologous plasma, autologous adipose tissue liposuction aspirate, allogeneic adipose tissue liposuction aspirate, allogeneic bone marrow aspirate, allogeneic blood, allogeneic buffy coat cells, allogeneic plasma, xenogeneic bone marrow aspirate, xenogeneic blood, xenogeneic adipose tissue aspirate, saline, and mixtures thereof.
